(19) United States
(12) Patent Application Publication
(10) Pub. No.: US 2026/0315798 A1
(43) Pub. Date: Oct. 15, 2026

(54) SYSTEM AND METHOD FOR A SELF-POWERED TOTALLY IMPLANTABLE COCHLEAR IMPLANT

(71) Applicants: INDIAN INSTITUTE OF TECHNOLOGY GUWAHATI, ASSAM (IN); INVITREO BIOSCIENCES PTE. LTD., Singapore (SG)

(72) Inventors: Kishore Kumar PADI, ASSAM (IN); Raagdeep RAJ, ASSAM (IN); Kanagaraj SUBRAMANI, ASSAM (IN)

(73) Assignees: INDIAN INSTITUTE OF TECHNOLOGY GUWAHATI, ASSAM (IN); INVITREO BIOSCIENCES PTE. LTD., Singapore (SG)

(21) Appl. No.: 19/299,554

(22) Filed: Aug. 14, 2025

(30) Foreign Application Priority Data
Apr. 9, 2025 (IN) 202531034606

(51) Int. Cl. A61N 1/36; A61N 1/378

(52) U.S. Cl. CPC A61N 1/36039; A61N 1/3785

(57) ABSTRACT

The present invention provides a system and method for a self-powered, totally implantable cochlear implant. The system comprises a Cantilever Beam Array (CBA) with piezoelectric cantilevers for frequency-specific sound detection, a Control Unit for analog signal processing and dynamic signal modulation, and an Electrode Array for precise auditory nerve stimulation. The system operates autonomously, powered by energy generated from the CBA, eliminating the need for external power sources. Advanced analog processing provides high-fidelity sound perception with minimal latency and distortion, while a real-time feedback mechanism allows adaptive adjustments for improved performance. The compact and lightweight design enables seamless integration inside the ear, offering customizable auditory rehabilitation tailored to individual needs. This invention addresses the limitations of existing cochlear implants, delivering a transformative solution for individuals with hearing impairments.

### TECHNICAL FIELD

The present invention is generally related to the field of biomedical engineering. The present invention is particularly related to auditory rehabilitation devices. The present invention is more particularly related to a system and method for a self-powered, totally implantable cochlear implant.

### BACKGROUND OF THE INVENTION

A significant portion of the population experiences hearing loss, with millions affected in India alone. Hearing loss can range from mild to profound and can affect both children and adults. Sensorineural hearing loss results from damage to the hair cells in the cochlea or the auditory nerve. Treatment of sensorineural hearing loss requires cochlear implants.

Conventional hearing aids typically use a microphone to pick up sound, which may have limitations, such as issues with water exposure. Existing treatment options include medical or surgical interventions, middle ear implants, and cochlear implants. Cochlear implants work by stimulating the auditory nerves. Current cochlear implant technology uses electrical pulses to stimulate the auditory nerve and requires an external power source.

Hence, there exists a need for a system and method for a self-powered, totally implantable cochlear implant that can detect sound efficiently and operate with low power.

The above-mentioned shortcomings, disadvantages and problems are addressed herein, which will be understood by reading and studying the following specification.

### OBJECT OF THE INVENTION

The primary object of the present invention is to provide a system and method for a self-powered, totally implantable cochlear implant replacing the function of a damaged inner ear.

Another object of the present invention is to eliminate the need for external power sources through self-powered operation.

Yet another object of the present invention is to achieve an amplitude control for auditory stimulation without the requirement of an external power source.

Yet another object of the present invention is to develop an analog control unit to customise and calibrate electrical pulses reaching cochlea from the piezoelectric sensor.

Yet another object of the present invention is to design low-power cochlear implants using a single chip that can be fully implanted, including the acoustic sensor.

Yet another object of the present invention is to detect the mechanical motion in the middle ear and convert them into electrical signals for processing.

Yet another object of the present invention is to achieve precise cochlear tonotopy replication for natural auditory nerve stimulation.

Yet another object of the present invention is to offer customizable auditory rehabilitation tailored to meet individual hearing profiles.

Yet another object of the present invention is to provide adjustments through a feedback mechanism for enhanced performance.

Yet another object of the present invention is to deliver high-fidelity sound perception with minimal latency and distortion.

These and other objects and advantages of the present invention will become readily apparent from the following detailed description taken in conjunction with the accompanying drawings.

### SUMMARY OF THE INVENTION

The various embodiments of the present invention provide a system and method for a self-powered, totally implantable cochlear implant.

According to one embodiment of the present invention, a system is provided for a self-powered, totally implantable cochlear implant. The system comprises a Cantilever Beam Array (CBA) designed to detect sound by converting mechanical vibrations of the ear into electrical signals; a control unit designed for analog signal processing and regulating the electrical signals received from CBA; and an electrode array implanted in the cochlea, configured to receive processed signals from the control unit and convert them into electrical pulses to stimulate the auditory nerve. The CBA is designed to be mounted completely inside the ear at locations where the mechanical signal transduction is maximized. The CBA converts the mechanical movement, induced by sound waves, into electrical signals, further transmitting them to the control unit. The CBA is designed to detect a broad range of sound frequencies, mimicking the natural frequency mapping (tonotopy) of the cochlea. The CBA comprises a plurality of piezoelectric cantilevers of varying precisely engineered lengths, each fine-tuned to resonate at specific frequencies, enabling frequency selective detection of sound. The cantilever generates electrical signal in response to the mechanical vibration. The piezoelectric cantilever is configured with a bottom electrode; a piezoelectric material; a top electrode; and a proof mask. The piezo electric sensors are designed to be made with a plurality of materials including PZT, AlN, ZnO, PVDF or a combination thereof. The control unit processes the electrical signals received from the CBA using analog signal processing techniques including amplification, filtering and frequency matching operations. The control unit assigns the processed signals to the corresponding nodes in the electrode array enabling precise stimulation of cochlea. The control unit is designed to precisely map the frequency and maintain the cochlear tonotopy, where the lower frequencies are directed to the apex of the cochlea and the higher frequencies to the base, replicating the natural hearing process. The control unit is precisely designed to be mounted on a stable structure near the middle ear, avoiding any direct connection with the ear's moving parts, and is powered by the CBA. The control unit comprises an amplitude control unit, regulating the strength of the electrical signals generated by the CBA, aligning the signals within the stimulation thresholds required for activating the auditory nerves. The amplitude control unit is designed as an entirely analog system, providing low latency and consistent performance with high reliability, preserving the signal integrity while maintaining the natural dynamics of sound perception. The analog design enables seamless transitions among varying sound levels, minimizes distortion and improves the overall auditory experience. According to one embodiment of the present invention, the amplitude control unit further comprises microswitches providing signal routing and modulation; and a switch matrix dynamically enhancing the signal amplification control. The micro switches manage the input form the CBA, wherein the switches selectively connect and disconnect cantilever outputs to the processing circuits. The microswitches are configured for signal selection, allowing specific frequency channels to be processed as required. The microswitches are configured to provide tailored auditory stimulation and optimize power efficiency by controlling individual cantilevers within a channel. The switch matrix provides for the signal amplification control by adjusting the connectivity based on the amplification needed to stimulate the auditory nerve, wherein weak signals from lower amplitude vibrations are amplified by connecting a plurality of cantilevers from the same channel. The switch matrix reduces the distortion and over stimulation due to stronger signals by bypassing the additional amplification, and thereby providing an optimal signal modulation across a range of auditory conditions. The microswitches are configured to be integrated with analog multiplexers providing additional layer of control by gating specific signals before providing to multiplexer, enhancing overall system flexibility. The multiplexer acts as a signal selector enabling a plurality of input signals from the CBA to share a single processing pathway, optimizing the resource utilization while providing accurate stimulation of the auditory nerve. The multiplexer selects and processes signals from different cantilevers within the CBA, prioritizing inputs based on predefined criteria including frequency, amplitude or clinical requirements. The multiplexer is configured during calibration to adjust switching patterns based on input conditions or auditory nerve responses, providing consistent and adaptive performance using feedback mechanisms and minimizing power consumption by processing only active channels. The multiplexer reduces noise and interference by selectively routing relevant signals, enabling clear transmission to the electrode array, and wherein the integration of the multiplexer enables scalability for implants with higher channel counts, providing more precise stimulation and improved replication of natural hearing. The electrode array is designed for implanting inside the cochlea receives the signal from the control unit and converts them into electrical pulses simulating the auditory nerve, replicating the function of the non-functional hair cells in the cochlea. The control unit provides precise calibration of the stimulation matching the intensity and the frequency of the original sound enabling the subject to perceive the sound with high fidelity. The feedback from the electrode array is used to adjust the microswitch configuration providing consistent performance across a plurality of environmental conditions and user requirement. The feedback configuration enables tuning and calibrating the device based on patient's auditory profile.

According to one embodiment of the present invention, a method is provided for auditory rehabilitation using a self-powered, totally implantable cochlear implant system, the method comprising: Detecting mechanical vibrations within the ear using a CBA; Generating electrical signals from the detected vibrations, wherein the piezoelectric cantilevers convert mechanical vibrations into frequency-specific electrical signals while simultaneously generating power for the system; Transmitting the electrical signals to a control unit, wherein the control unit is configured to process and regulate the received signals for auditory stimulation; Processing the electrical signals in the control unit, including amplifying and modulating the electrical signals using a switch matrix and microswitches; Delivering the processed electrical signals to an electrode array implanted within the cochlea; and Stimulating the auditory nerve with electrical pulses, wherein the stimulation replicates the natural frequency mapping of the cochlea, enabling high-fidelity sound perception. The method for processing the electrical signals in the control unit further comprises: Amplifying the electrical signals to align with stimulation thresholds of the auditory nerve, providing sufficient signal strength for effective nerve activation; Filtering out noise and non-relevant components from the signals to maintain clarity and integrity; Performing frequency mapping to assign the signals to specific channels corresponding to the cochlear tonotopy, replicating the natural frequency organization of the cochlea; modulating the signals using an amplitude control unit, wherein signal strength is adjusted based on real-time auditory conditions; signal routing and amplification, enabling selective activation of cantilever outputs using microswitches; Signal selection and routing using a switch matrix, enabling efficient processing of multiple input signals while maintaining energy efficiency; and Communicating processed signals to the electrode array for precise auditory nerve stimulation, enhancing auditory perception and fidelity. The method for processing the electrical signals further comprises: Sharing processing pathways among multiple input signals from the cantilever beam array, thereby reducing hardware complexity and enhancing compactness and energy efficiency; Selecting and routing signals based on predefined criteria, including signal frequency, amplitude, and contextual auditory conditions; Prioritizing signals from specific cantilevers during calibration using the multiplexer, enhancing frequencies corresponding to the patient's unique hearing profile; Combining outputs from multiple cantilevers within a frequency channel amplifying weak signals while bypassing amplification for stronger signals, prevent overstimulation; Reducing noise and interference by selectively routing relevant signals; Scaling the system by accommodating higher channel counts, enabling precise auditory nerve stimulation across a broader frequency spectrum; and Adapting signal routing based on feedback from the auditory nerve, for consistent performance and personalized auditory rehabilitation.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a schematic cross-section of the human ear.

FIG. 2 illustrates an overall block diagram of the system for a self-powered, totally implantable cochlear implant, according to one embodiment of the present invention.

FIG. 3a illustrates the Cantilever Beam Array (CBA), according to one embodiment of the present invention.

FIG. 3b illustrates the arrangement of cantilevers within the CBA, according to one embodiment of the present invention.

FIG. 4 illustrates a piezoelectric cantilever beam, according to one embodiment of the present invention.

FIG. 5 illustrates a Control Unit combined with the CBA, according to one embodiment of the present invention.

FIG. 6 illustrates microswitches in the amplitude control unit within the control system, according to one embodiment of the present invention.

FIG. 7(a) illustrates audiogram of a patient with severe to profound hearing loss in 3000 Hz to 8000 Hz frequency range in left ear

FIG. 7(b) illustrates an example embodiment of the device configured to compensate for hearing loss in the patient, according to one embodiment of the present invention.

FIG. 8(a) illustrates an independent output response of a piezoelectric sensor in the CBA designed to achieve maximum output at 3000 Hz, according to one embodiment of the present invention.

FIG. 8(b) illustrates the combined output response of two piezoelectric sensors in the same channel, indicating linear addition and enhanced signal amplitude, according to one embodiment of the present invention.

FIG. 9(a) illustrates an independent output response of a piezoelectric sensor optimized for 8000 Hz, according to one embodiment of the present invention.

FIG. 9(b) illustrates the aggregated response of four piezoelectric sensors within the same channel at 8000 Hz, indicating signal amplification through linear addition., according to one embodiment of the present invention

FIG. 10 illustrates a method for a self-powered, totally implantable cochlear implant, according to one embodiment of the present invention.

Although the specific features of the present invention are shown in some drawings and not in others. This is done for convenience only as each feature may be combined with any or all of the other features in accordance with the present invention.

### DETAILED DESCRIPTION OF THE INVENTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that other changes may be made without departing from the scope of the embodiments. The following detailed description is therefore not to be taken in a limiting sense.

The various embodiments of the present invention provide a system and method for a self-powered, totally implantable cochlear implant.

According to one embodiment of the present invention, a system is provided for a self-powered, totally implantable cochlear implant. The system comprises a CBA designed to detect sound by converting mechanical vibrations into electrical signals; a control unit designed for analog signal processing and regulating the electrical signals received from CBA; and an electrode array implanted in the cochlea, configured to receive processed signals from the control unit and convert them into electrical pulses to stimulate the auditory nerve.

According to one embodiment of the present invention, the CBA is designed to be implanted inside the ear at locations targeted for maximizing the mechanical signal transduction. The CBA converts the mechanical movement, induced by sound waves, into electrical signals, further transmitting them to the control unit. The CBA is designed to detect a broad range of sound frequencies, mimicking the natural frequency mapping (tonotopy) of the cochlea.

According to one embodiment of the present invention, the CBA comprises a plurality of piezoelectric cantilevers of varying precisely engineered lengths, each fine-tuned to resonate at specific frequencies, enabling frequency selective detection of sound. The cantilever generates electrical signal in response to the mechanical vibrations.

According to one embodiment of the present invention, a piezoelectric cantilever is configured with a bottom electrode; a piezoelectric material and a top electrode; along with a proof mask. The piezo electric sensors are designed to be made with a plurality of materials including PZT, AlN, ZnO, PVDF or a combination thereof.

According to one embodiment of the present invention, the control unit processes the electrical signals received from the CBA using analog signal processing techniques including amplification, filtering and frequency matching operations. The control unit assigns the processed signals to the corresponding nodes in the electrode array enabling precise stimulation of cochlea. The control unit is designed to precisely map the frequency and maintain the cochlear tonotopy, where the lower frequencies are directed to the apex of the cochlea and the higher frequencies to the base, replicating the natural hearing process.

According to one embodiment of the present invention, the control unit is precisely designed to be mounted on a stable structure near the middle ear, avoiding any direct connection with the ear's moving parts, and is powered by the CBA.

According to one embodiment of the present invention, the control unit comprises an amplitude control unit, regulating the strength of the electrical signals generated by the CBA, aligning the signals within the stimulation thresholds required for activating the auditory nerves. The amplitude control unit is designed as an entirely analog system, providing low latency and consistent performance with high reliability, preserving the signal integrity while maintaining the natural dynamics of sound perception. The analog design enables seamless transitions between varying sound levels, minimizes distortion and improves the overall auditory experience.

According to one embodiment of the present invention, the amplitude control unit further comprises microswitches providing signal routing and modulation; a switch matrix enhancing the signal amplification control;

According to one embodiment of the present invention, the micro switches manage the input form the CBA, wherein the switches selectively connect and disconnect cantilever outputs to the processing circuits. The microswitches are configured for signal selection, allowing specific frequency channels to be processed as required. The microswitches are configured to provide tailored auditory stimulation and optimize power efficiency by controlling individual cantilevers within a channel.

According to one embodiment of the present invention, the switch matrix enhances the signal amplification control, by adjusting the connectivity based on the amplification needed of the subject to stimulate the auditory nerve, wherein weak signals from lower amplitude vibrations are amplified by connecting a plurality of cantilevers from the same channel. The switch matrix reduces the distortion and over stimulation due to stronger signals by bypassing the additional amplification and thereby providing an optimal signal modulation across a range of auditory conditions.

According to one embodiment of the present invention, the microswitches are configured to be integrated with analog multiplexers providing additional layer of control by gating specific signals before providing to multiplexer, enhancing overall system flexibility. The multiplexer acts as a signal selector enabling a plurality of input signals from the CBA to share a single processing pathway, optimizing the resource utilization while providing accurate stimulation of the auditory nerve. The multiplexer selects and processes signals from different cantilevers within the CBA, prioritizing inputs based on predefined criteria including frequency, amplitude or clinical requirements. The multiplexers are designed to switch between input signals during calibration and tuning, maintaining comprehensive coverage of the auditory spectrum. The multiplexer is further configured to adjust switching patterns in based on input conditions or auditory nerve responses, providing consistent and adaptive performance using feedback mechanisms.

According to one embodiment of the present invention, the multiplexers switch rapidly between input signals, maintaining processing and comprehensive coverage of the auditory spectrum. The multiplexer is further configured to adjust switching patterns based on input conditions or auditory nerve responses, providing consistent and adaptive performance using feedback mechanisms, and minimizing power consumption by processing only active channels.

According to one embodiment of the present invention, the multiplexer reduces noise and interference by selectively routing relevant signals, enabling clear transmission to the electrode array, and wherein the integration of the multiplexer enables scalability for implants with higher channel counts, providing more precise stimulation and improved replication of natural hearing.

According to one embodiment of the present invention, the electrode array is designed for implanting inside the cochlea receives the signal from the control unit and converts them into electrical pulses stimulating the auditory nerve, replicating the function of the non-functional hair cells in the cochlea. The control unit provides precise calibration of the stimulation matching the intensity and the frequency of the original sound enabling the subject to perceive the sound with high fidelity.

According to one embodiment of the present invention, the feedback from the electrode array is used to adjust the microswitch configuration providing consistent performance across a plurality of environmental conditions and user activity. The feedback configuration enables tuning and calibrating the device based on patient's auditory profile.

According to one embodiment of the present invention, a method is provided for auditory rehabilitation using a self-powered, totally implantable cochlear implant system, the method comprising: Detecting mechanical vibrations inside the ear using a cantilever beam array; Generating electrical signals from the detected vibrations, wherein the piezoelectric cantilevers convert mechanical vibrations into frequency-specific electrical signals while simultaneously generating power for the system; Transmitting the electrical signals to a control unit, wherein the control unit is configured to process and regulate the received signals for auditory stimulation; Processing the electrical signals in the control unit, including amplifying the signals, filtering noise, and performing frequency mapping; Routing and modulating the electrical signals using a switch matrix and microswitches; Delivering the processed electrical signals to an electrode array implanted within the cochlea; and Stimulating the auditory nerve with electrical pulses, wherein the stimulation replicates the natural frequency mapping of the cochlea, enabling high-fidelity sound perception.

According to one embodiment of the present invention, the method for processing the electrical signals in the control unit further comprises: Amplifying the electrical signals to align with stimulation thresholds of the auditory nerve, providing sufficient signal strength for effective nerve activation; Filtering out noise and non-relevant components from the signals to maintain clarity and integrity; Performing frequency mapping to assign the signals to specific channels corresponding to the cochlear tonotopy, replicating the natural frequency organization of the cochlea; modulating the signals using an amplitude control unit, wherein signal strength is adjusted based on auditory conditions; Signal routing and amplification, enabling selective activation of cantilever outputs using microswitches; Signal selection and routing using a switch matrix, enabling efficient processing of multiple input signals while maintaining energy efficiency; and Communicating processed signals to the electrode array for precise auditory nerve stimulation, enhancing auditory perception and fidelity.

According to one embodiment of the present invention, the method for processing the electrical signals further comprises: Sharing processing pathways among multiple input signals from the cantilever beam array, thereby reducing hardware complexity and enhancing compactness and energy efficiency; Selecting and routing signals based on predefined criteria, including signal frequency, amplitude, and contextual auditory conditions; Prioritizing signals from specific cantilevers during calibration using the multiplexer, enhancing frequencies corresponding to the patient's unique hearing profile; Combining outputs from multiple cantilevers within a frequency channel amplifying weak signals while bypassing amplification for stronger signals, prevent overstimulation; Reducing noise and interference by selectively routing relevant signals; Scaling the system by accommodating higher channel counts, enabling precise auditory nerve stimulation across a broader frequency spectrum; and Adapting signal routing based on feedback from the auditory nerve, for consistent performance and personalized auditory rehabilitation.

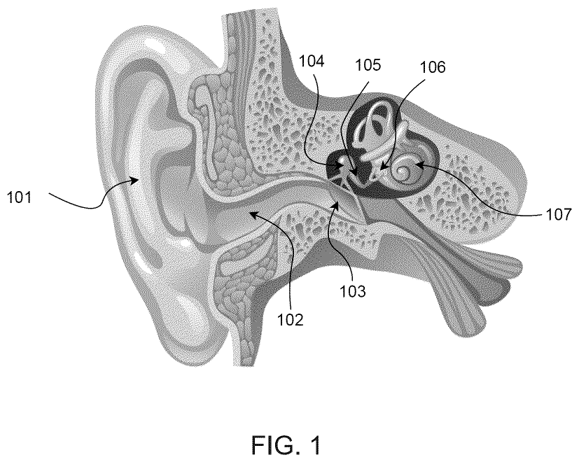

FIG. 1 illustrates a schematic cross-section of the human ear comprising: Outer Ear which includes the pinna (101), ear canal (102), and tympanic membrane (103), Middle Ear which includes the malleus (104), incus (105), and stapes (106), and Inner Ear which includes the cochlea (107).

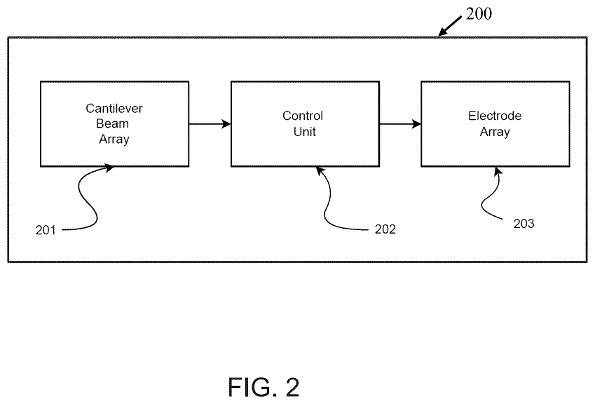

FIG. 2 illustrates an overall block diagram of the system for a self-powered, totally implantable cochlear implant, according to one embodiment of the present invention. The system comprises: Cantilever Beam Array 201; Control Unit 202; and Electrode Array 203

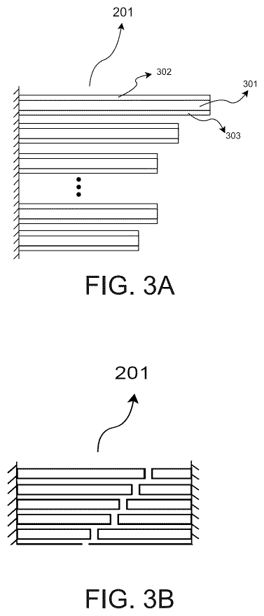

FIG. 3a illustrates the Cantilever Beam Array (CBA), according to one embodiment of the present invention. The CBA comprises layer of piezoelectric material 301, one layer of the top electrode 302 and one layer of the bottom electrode 303.

FIG. 3b illustrates the arrangement of cantilevers within the CBA, according to one embodiment of the present invention.

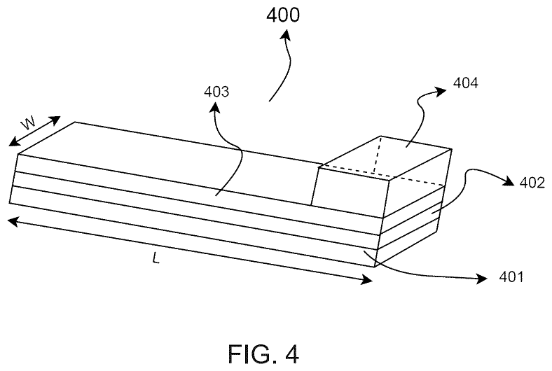

FIG. 4 illustrates a piezoelectric cantilever beam, according to one embodiment of the present invention. The piezoelectric cantilever beam comprises the bottom electrode 401, piezoelectric materials 402, a top electrode 403, and Proof mask 404.

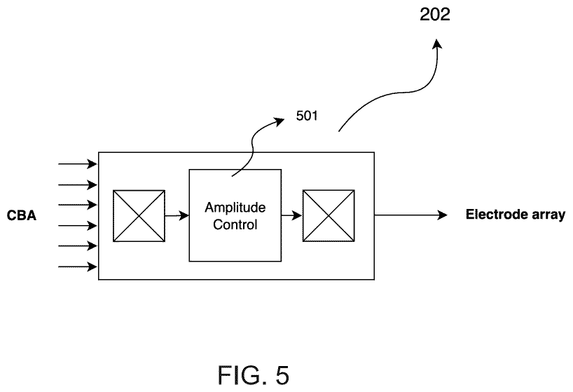

FIG. 5 illustrates a schematic of Control Unit 202 comprising an amplitude control unit 501, according to one embodiment of the present invention.

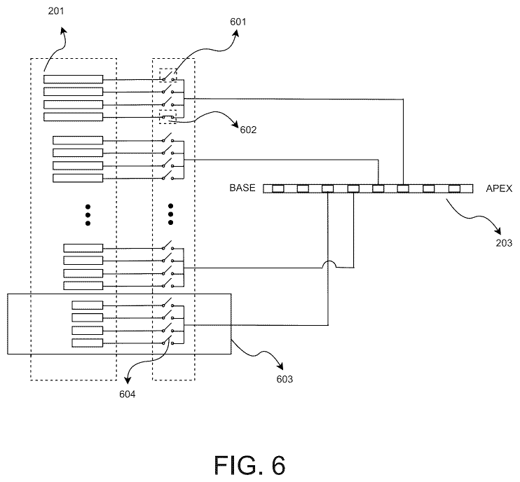

FIG. 6 illustrates microswitches in the amplitude control unit within the control system, according to one embodiment of the present invention. The system indicates micro switches in Open state 601; Closed state 602; and switches in a switch matrix 603 and 604.

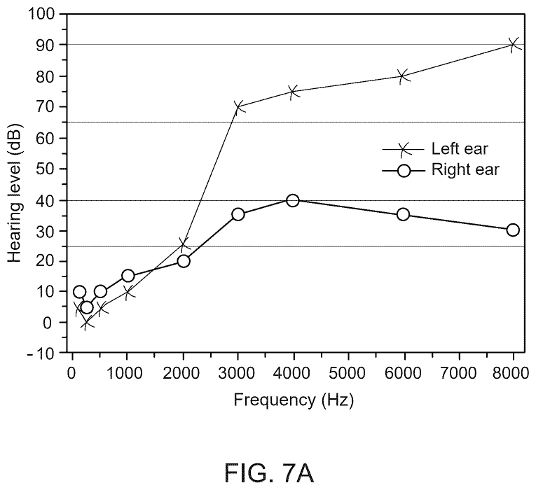

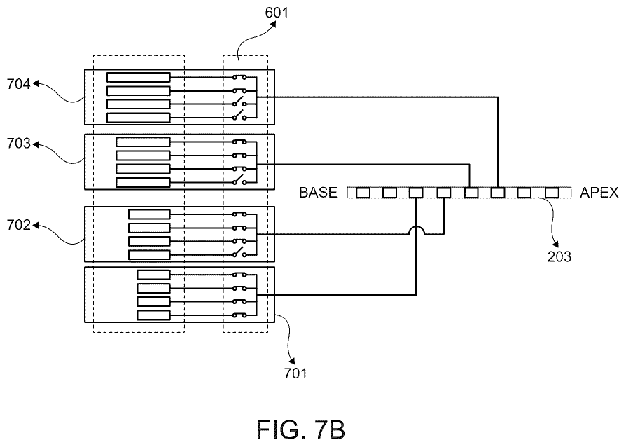

FIG. 7(a) illustrates audiogram of a patient with severe to profound hearing loss in left ear in the frequency range of 3000 Hz to 8000 Hz.

FIG. 7(b) illustrates an example embodiment of the device configured to compensate for hearing loss in the patient, according to one embodiment of the present invention. The system comprising microswitches 601 and four separate channels 701, 702, 703 and 704.

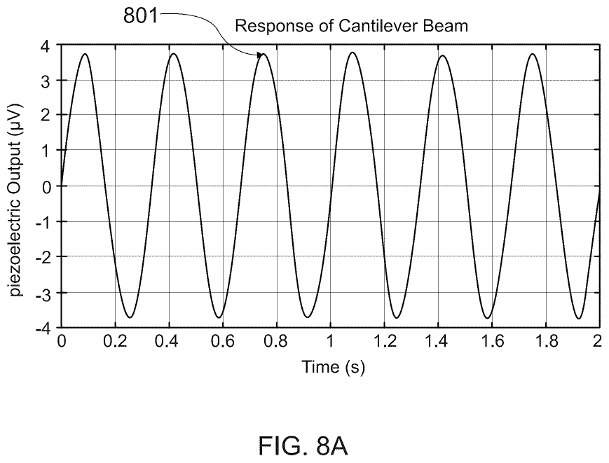

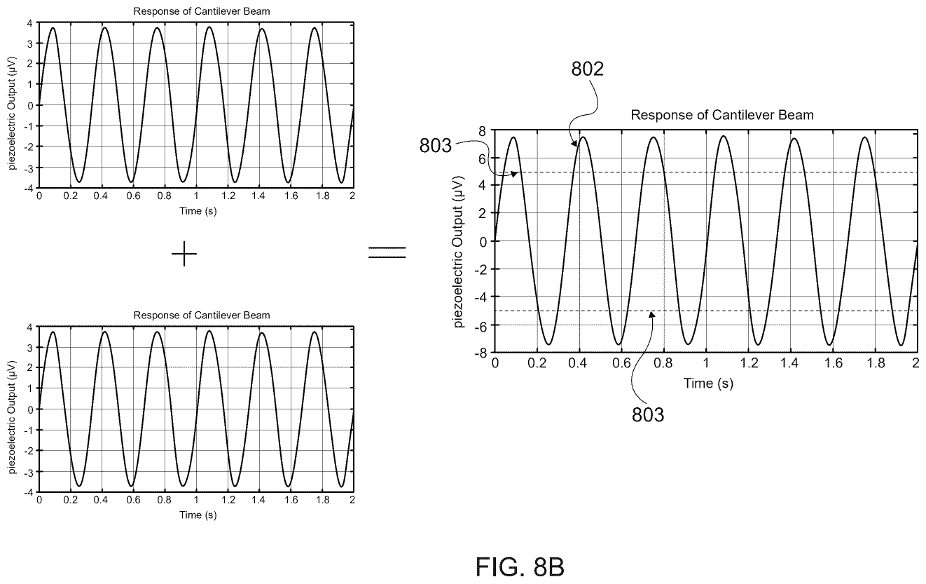

FIG. 8(a) illustrates an independent output response of a piezoelectric sensor in the CBA designed to achieve maximum output at 3000 Hz, according to one embodiment of the present invention. The response comprises a peak response of 3.73 μV, 801, at a resonance frequency of 3000 Hz.

FIG. 8(b) illustrates the combined output response of two piezoelectric sensors in the same channel, indicating linear addition and enhanced signal amplitude, according to one embodiment of the present invention. The response indicates aggregated signal produced by the two piezoelectric sensors 802, and the threshold level required to stimulate the auditory nerves in the subject 803.

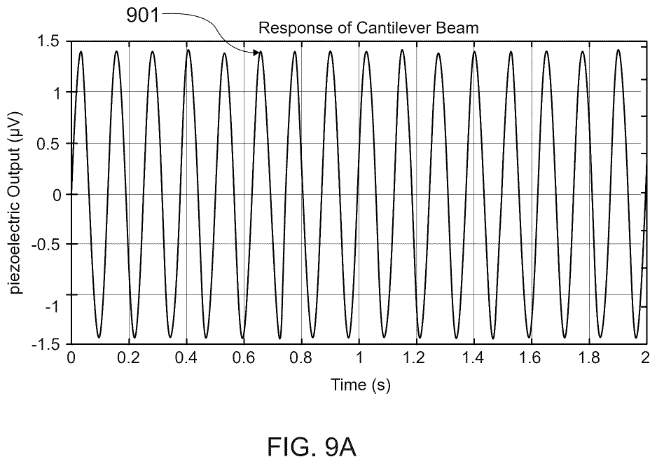

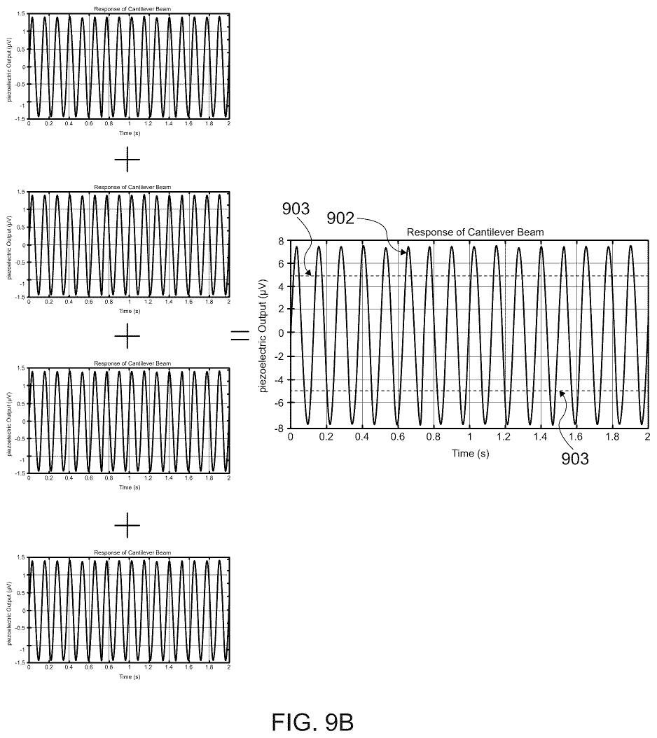

FIG. 9(a) illustrates an independent output response of a piezoelectric sensor optimized for 8000 Hz, according to one embodiment of the present invention. The response indicates a peak response of 1.47 μV, 901, at this a resonance frequency of 8000 Hz.

FIG. 9(b) illustrates the aggregated response of four piezoelectric sensors within the same channel at 8000 Hz, indicating signal amplification through linear addition., according to one embodiment of the present invention. The plot indicates aggregated response of the four sensors 902, and the threshold level required for auditory nerve stimulation in the subject 903.

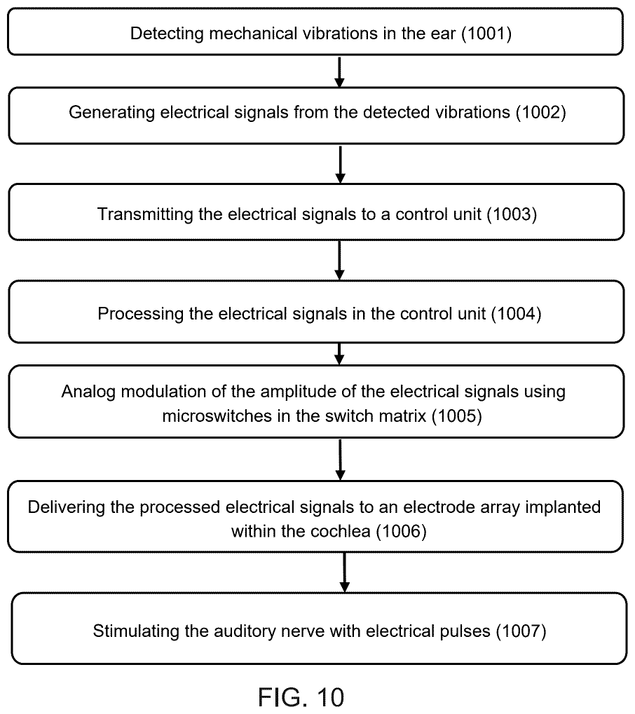

FIG. 10 illustrates a method for a self-powered, totally implantable cochlear implant, according to one embodiment of the present invention. The method comprises: Detecting mechanical vibrations in the ossicles (1001); Generating electrical signals from the detected vibrations (1002); Transmitting the electrical signals to a control unit (1003); Processing the electrical signals in the control unit (1004); Routing and modulating the electrical signals using a switch matrix and microswitches (1005); Delivering the processed electrical signals to an electrode array implanted within the cochlea (1006); and Stimulating the auditory nerve with electrical pulses (1007)

Although the embodiments herein are described with various specific embodiments, it will be obvious for a person skilled in the art to practice the embodiments herein with modifications.

### Advantages of the Invention

The various embodiments of the present invention provide a system and method for a self-powered, totally implantable cochlear implant. By utilizing piezoelectric cantilevers for sound detection and energy generation, the system eliminates the need for external power sources, providing enhanced autonomy and convenience for users.

The system delivers high-fidelity auditory rehabilitation through frequency-selective sound detection and precise cochlear tonotopy replication, enabling natural sound perception. By leveraging analog signal processing, the system achieves energy efficiency while minimizing latency and distortion, offering reliable and seamless auditory stimulation.

The compact and lightweight design of the system enables implantation inside the ear without interfering with natural ear functions, providing user comfort and ease of integration. The system also incorporates a feedback mechanism, enabling adaptive adjustments to improve performance under varying auditory conditions.

The customizable nature of the system allows clinicians to prioritize specific frequencies based on user needs, making it suitable for diverse hearing profiles. These advantages make the present invention a transformative solution for auditory rehabilitation, addressing the limitations of existing cochlear implant technologies.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

We claim:

1. A system for a self-powered, totally implantable cochlear implant, the system comprising:
a Cantilever Beam Array (CBA), wherein the Cantilever Beam Array is configured to convert mechanical vibrations from the ossicles into electrical signals;
a Control Unit, wherein Control Unit is an analog system designed to process and regulate the electrical signals received from CBA, enabling alignment with auditory stimulation thresholds; and
an Electrode Array, wherein the Electrode Array implanted in the cochlea, is configured to receive processed signals from the control unit and convert them into electrical pulses to stimulate the auditory nerve.

2. The system as claimed in claim 1, wherein the Cantilever Beam Array (CBA) designed to be placed inside the ear at specific locations targeted to maximizing the mechanical signal transduction, converts the mechanical movement, induced by sound waves, into electrical signals, and transmits them to the control unit, wherein The cantilever beam array is designed to detect a broad range of sound frequencies, mimicking the natural frequency mapping (tonotopy) of the cochlea, wherein the cantilever beam array comprises a plurality of piezoelectric cantilevers of varying precisely engineered lengths, each resonating at specific frequencies, enabling frequency selective detection of sound; and wherein the piezoelectric cantilever configured with a bottom electrode, a piezoelectric material, a top electrode, and a proof mask, is designed to be constructed with a plurality of materials including PZT, AlN, ZnO, PVDF or a combination thereof.

3. The system as claimed in claim 1, wherein the control unit processes the electrical signals received from the CBA using analog signal processing techniques including amplification, filtering and frequency matching operations, wherein the control unit assigns the processed signals to the corresponding nodes in the electrode array enabling precise stimulation of cochlea, mapping the frequency and maintain the tonotopy of the cochlea, where the lower frequencies are directed to the apex of the cochlea and the higher frequencies to the base, replicating the natural hearing process, wherein the control unit is designed to be mounted on a stable structure near the middle ear, avoiding any direct connection with the ear's moving parts, and is powered by the cantilever beam array.

4. The system as claimed in claim 1, wherein the control unit comprises an amplitude control unit, regulating the strength of the electrical signals generated by the CBA, aligning the signals within the stimulation thresholds required for activating the auditory nerves, wherein the amplitude control unit is designed as an entirely analog system, providing low latency and consistent performance with high reliability, preserving the signal integrity while maintaining the natural dynamics of sound perception, enabling seamless transitions between varying sound levels, minimizing distortion and improving the overall auditory experience.

5. The system as claimed in claim 1, wherein the amplitude control unit further comprises microswitches providing signal routing and modulation; a switch matrix enhancing the signal amplification control; wherein the micro switches manage the input from the CBA, selectively connecting and disconnecting cantilever outputs to the processing circuits, wherein the microswitches are configured for signal selection, allowing specific frequency channels to be processed as required, providing tailored auditory stimulation and optimizing power efficiency by controlling individual cantilevers within a channel; wherein the switch matrix enhances the signal amplification control by adjusting the connectivity based on the amplification needed to stimulate the auditory nerve, wherein weak signals from lower amplitude vibrations are amplified by connecting a plurality of cantilevers from the same channel, while bypassing the stronger signals reducing the distortion and over stimulation, and thereby providing an optimal signal modulation across a range of auditory conditions.

6. The system as claimed in claim 1, wherein the microswitches are configured to be integrated with analog multiplexers providing additional layer of control by gating specific signals before providing to multiplexer, enhancing overall system flexibility, wherein the multiplexer acts as a signal selector enabling a plurality of input signals from the CBA to share a single processing pathway, optimizing the resource utilization while providing accurate stimulation of the auditory nerve, wherein the multiplexer selects and processes signals from different cantilevers within the CBA, prioritizing inputs based on predefined criteria including frequency, amplitude or clinical requirements, wherein the multiplexers switch rapidly between input signals, maintaining configurability in processing and comprehensive coverage of the auditory spectrum, and wherein the multiplexer is further configured to adjust switching patterns in real-time based on input conditions or auditory nerve responses, providing consistent and adaptive performance using feedback mechanisms, wherein the multiplexer minimizes power consumption by processing only active channels, wherein the multiplexer reduces noise and interference by selectively routing relevant signals, enabling clear transmission to the electrode array, and wherein the integration of the multiplexer enables scalability for implants with higher channel counts, providing more precise stimulation and improved replication of natural hearing.

7. The system as claimed in claim 1 wherein, the electrode array implanted inside the cochlea receives the signal from the control unit and converts them into electrical pulses simulating the auditory nerve, replicating the function of the non-functional hair cells in the cochlea, wherein the control unit provides precise calibration of the stimulation matching the intensity and the frequency of the original sound enabling the subject to perceive the sound with high fidelity, wherein the feedback from the electrode array is used to adjust the microswitch configuration providing consistent performance across a plurality of environmental conditions and user activity, enabling tuning and calibrating the device based on patient's auditory profile.

8. A method for providing auditory rehabilitation using a self-powered, totally implantable cochlear implant system, the method comprising:
detecting mechanical vibrations inside the ear using a cantilever beam array;
generating electrical signals from the detected vibrations, wherein the piezoelectric cantilevers convert mechanical vibrations into frequency-specific electrical signals while simultaneously generating power for the system;
transmitting the electrical signals to a control unit, wherein the control unit is configured to process and regulate the received signals for auditory stimulation;
processing the electrical signals in the control unit, including amplifying the signals aligning with the stimulation thresholds of the auditory nerve, filtering noise and irrelevant components for improved signal clarity, and performing frequency mapping assign signals to specific cochlear frequency channels based on tonotopy;
routing and modulating the electrical signals using a switch matrix and microswitches;
delivering the processed electrical signals to the electrode array implanted within the cochlea; and
stimulating the auditory nerve with electrical pulses, wherein the stimulation replicates the natural frequency mapping of the cochlea, enabling high-fidelity sound perception.

9. The method as claimed in claim 8, wherein processing the electrical signals in the control unit further comprises: Amplifying the electrical signals to align with stimulation thresholds of the auditory nerve, providing sufficient signal strength for effective nerve activation; Filtering out noise and non-relevant components from the signals to maintain clarity and integrity; Performing frequency mapping to assign the signals to specific channels corresponding to the cochlear tonotopy, replicating the natural frequency organization of the cochlea; Modulating the signals using an amplitude control unit, wherein signal strength is adjusted based on auditory conditions; Configurable signal routing and amplification, enabling selective activation of cantilever outputs using microswitches Signal selection and routing using a switch matrix, enabling efficient processing of multiple input signals while maintaining energy efficiency; and, Communicating processed signals to the electrode array for precise auditory nerve stimulation, enhancing auditory perception and fidelity.

10. The method as claimed in claim 8, wherein processing the electrical signals further comprises: Sharing processing pathways among multiple input signals from the cantilever beam array, thereby reducing hardware complexity and enhancing compactness and energy efficiency; Selecting and routing signals based on predefined criteria, including signal frequency, amplitude, and contextual auditory conditions; Prioritizing signals from specific cantilevers during calibration using the multiplexer, enhancing frequencies corresponding to the patient's unique hearing profile; Combining outputs from multiple cantilevers within a frequency channel amplifying weak signals while bypassing amplification for stronger signals, prevent overstimulation; Reducing noise and interference by selectively routing relevant signals; Scaling the system by accommodating higher channel counts, enabling precise auditory nerve stimulation across a broader frequency spectrum; and Adapting signal routing in based on feedback from the auditory nerve, for consistent performance and personalized auditory rehabilitation.
